(19) United States
(12) Patent Application Publication
(10) Pub. No.: US 2026/0263204 A1
(43) Pub. Date: Sep. 10, 2026

(54) CONTROLLED RELEASE INSEMINATION DEVICE, SYSTEM AND METHOD THEREOF

(71) Applicant: INTIMO MEDICAL LTD., Nazareth (IL)

(72) Inventor: Matan SHABAT, Eilat (IL)

(21) Appl. No.: 18/873,489

(22) PCT Filed: Jun. 12, 2023
(86) PCT No.: PCT/IL2023/050605

§ 371 Date: Dec. 10, 2024

(30) Foreign Application Priority Data
Jun. 12, 2022 (IL) 293854

(51) Int. Cl. A61D 19/02

(52) U.S. Cl. CPC A61D 19/028

(57) ABSTRACT

A device, system and method for intrauterine delivery of fluids to a uterus of a human or animal body wherein the delivery of the insemination fluid is controllable.

### FIELD OF THE INVENTION

The present invention relates to a device and system for intrauterine delivery of fluids and in particular, to such a device and system providing controlled release of insemination fluids to a uterus.

### BACKGROUND OF THE INVENTION

Artificial insemination attempts to assist fertility in humans and/or control fertility in livestock by introducing an insemination fluid, namely, semen directly into the uterine cavity, generally near the opening of the fallopian tube. Such insemination is accomplished with a dedicated catheter known as an IntraUterine Insemination (‘IUI’) catheter. An IUI catheter introduced semen past the cervix so as to increase likelihood of in utero fertilization and may be applied both to humans or animals particularly livestock such as horses.

### SUMMARY OF THE INVENTION

The present invention overcomes the deficiencies of the background art by providing a system and device for improving the likelihood and/or improving the success rate of current insemination catheters by controlling at least one of the delivery rate and/or location.

Embodiments of the present invention provide a controlled release intrauterine insemination device and associated system. The controlled release insemination device may deliver the insemination fluid at different release rate and therefore may be configured to be both a Slow Release Insemination (SRI) device or a Bolus Insemination Device (IUI, BI) or a combination thereof or an intermittent release rate insemination device.

In embodiments the insemination fluid release rate may be controlled based on sensed conditions within the targeted delivery site.

In embodiments the delivery site within the uterus may be selected based on sensed conditions for example including but not limited to pH, temperature, visualized obstructions, the like or any combination thereof.

In embodiments the delivery site within the uterus may be selected based on sensed conditions for example including but not limited to pressure.

In embodiments the controlled release device according to embodiments of the present invention may be configured to be used for human and/or veterinary use. In embodiments the device may be utilized for insemination of livestock for example including but not limited to horses, cows, sheep, goat, bovine, buffalo, or the like. In embodiments, the device may be configured to be utilized for insemination of wild and/or endangered animals.

In embodiments, the controlled release insemination device may be configured to comprise a retentive module, a delivery module, a delivery channel, an electronics module, and a sensor module. In embodiments the controlled release insemination device may form a system comprising the device according to optional embodiments, that is configured to be in communication with a communication and processing auxiliary device.

In embodiments the auxiliary device may be in further communication with a data processing center.

Embodiments of the present invention provide a controlled release intrauterine insemination device, the device comprising, a retentive module, a delivery channel, a delivery module, an electronics module, and a sensor module, wherein the delivery module may be controlled based on data captured with the sensor module to control a delivery rate of a sample associated with the channel so as to determine sample delivery parameters.

In embodiments the retentive module may be an inflatable balloon or a stent-like expandable anchor. In some embodiments, the retentive module may comprise at least two or more retentive members. Optionally, the retentive members may for example be selected from an inflatable balloon or a stent-like anchor.

In embodiments, the retentive members may be disposed along different locations of the delivery channel.

In embodiments, a first retentive member may be disposed adjacent to a distal end of the delivery channel and a second retentive member may be disposed adjacent to a proximal end of the delivery channel. Optionally, the second retentive member may be configured in the form of a device housing. In embodiments, the second retentive member may be disposed proximal to a cervix. In embodiments, the first retentive member may be disposed distal to a cervix and the second retentive member may be disposed proximal to the cervix.

In some embodiments, wherein the delivery channel may be configured to have a distal end that may be distal to a distal end of a retentive module.

In embodiments, the delivery channel may be associated with at least one or more sensors of the sensor module. Optionally, the at least one or more sensors may be disposed along the delivery channel. In embodiments, the at least one or more sensors are disposed along an end of the delivery channel.

In embodiments the delivery channel sensor and/or sensor module may comprise a pressure sensor, preferably disposed about a leading edge and/or distal end of the delivery channel in order to facilitate placement. Preferably such a pressure sensor provides for ensuring that the placement is done accurately without perforating and/or damaging tissue at the delivery site. Preferably such a pressure sensor allows for limiting the force and/or pressure applied when placing the device.

In embodiments the delivery channel may feature a light source for facilitating placement of the device at the treatment site. Optionally the light source may be provided in optional forms for example including but not limited to a light emitting diode ‘LED’ disposed at a portion of the distal end of the delivery channel. Optionally the light source may be provided in the form of an optic fiber and associated light source such that the optic fiber tip is disposed at the distal end of the delivery channel while the associated light source is disposed at a proximal portion of the delivery channel.

In some embodiments, the sensor may be disposed adjacent to a distal end of the delivery channel.

In some embodiments the sensor may be disposed along a portion of a distal end of the delivery channel.

In embodiments, at least one or more sensors may be disposed along the length of the delivery channel.

In some embodiments two of the same sensor types may be disposed along two different locations along the delivery channel.

In embodiments, the delivery channel may be configured to feature at least two or more delivery ports.

In embodiments, at least one delivery port may be disposed along a distal end of the delivery channel. Optionally, a second delivery port may be disposed along the length of the delivery channel. Optionally, at least one or more sensors may be disposed adjacent to each of the delivery ports.

In embodiments, the sensor module may for example include but is not limited to at least one sensor selected from: image sensor, pH sensor, flow sensor, temperature sensor, pressure sensor, the like or any combination thereof.

In embodiments, the delivery module may be provided in the form of an electromechanical pump.

In embodiments, the delivery module may be configured to generate either positive pressure or negative pressure adjacent to the delivery port.

Embodiments of the present invention provide a controlled release intrauterine insemination system comprising the delivery device according to the present invention that is configured to be in wireless communication with an auxiliary processing and communication device.

In embodiments, the system may further comprise a data processing center in communication with the auxiliary device.

Embodiments of the present invention provide a method for controlling the delivery rate of an insemination sample with the delivery device and/or system according to embodiments of the present invention so as to determine the delivery parameters of an insemination sample, the parameters selected from at least one of: determining delivery site withing the intrauterine space; determining rate of sample delivery; determining the timing of sample delivery; determining the sample delivery ejection speed ; determining the sample delivery ejection force, the like or any combination thereof.

In embodiments, electronics module preferably comprises necessary electronic circuitry and processing hardware and/or software to render the IUI device functional.

In embodiments, delivery channel may be configured as a channel and/or a catheter for receiving a sample for delivery. In embodiments delivery channel may be configured to receive a disposable and/or single use catheter. In embodiments delivery channel may be configured to be a multi-use channel for directly receiving and delivery a semen sample.

In embodiments controlled release device may comprise a sensor module comprising at least one or more sensor to render the device functions. In embodiments sensor module may for example include at least one or more sensors for example including but not limited to: image sensor, pH sensor, flow sensor, temperature sensor, pressure sensor, positional sensor, the like or any combination thereof.

In embodiments controlled release device according to the present invention may be configured to communicate with an auxiliary device via wireless communication Data Processing Center provided to receive and process data relating to device and treatments thereof. For example, a data processing center may for example be utilized to perform big data analysis wherein such analysis may be utilized to improve success rates, to change treatment protocols, to undertake artificial intelligence (AI) analysis, to review all historic data. In some embodiments, data processing center may be utilized to provide advise so as to improve success rate, more preferably in substantially real time.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic block diagrams of an exemplary device and system according to embodiments of the present invention.

### DESCRIPTION OF THE PREFERRED EMBODIMENTS

The principles and operation of the present invention may be better understood with reference to the drawings and the accompanying description. The following figure reference labels are used throughout the description to refer to similarly functioning components are used throughout the specification hereinbelow.
50 Data Processing Center;
100 controlled release insemination device;
102 retentive module;
104 delivery module;
104a negative pressure sub-module;
104b positive pressure sub-module;
106 delivery channel;
106a delivery port;
110 electronics module;
111 user interface (UI);
112 power module;
114 controller and/or processor module;
116 communication module;
118 memory module;
120 sensor module;
121 pressure sensor;
122 temperature sensor;
123 light source;
124 pH sensor;
126 imaging sensor;
128 flow sensor;
130 auxiliary device;
150 system;

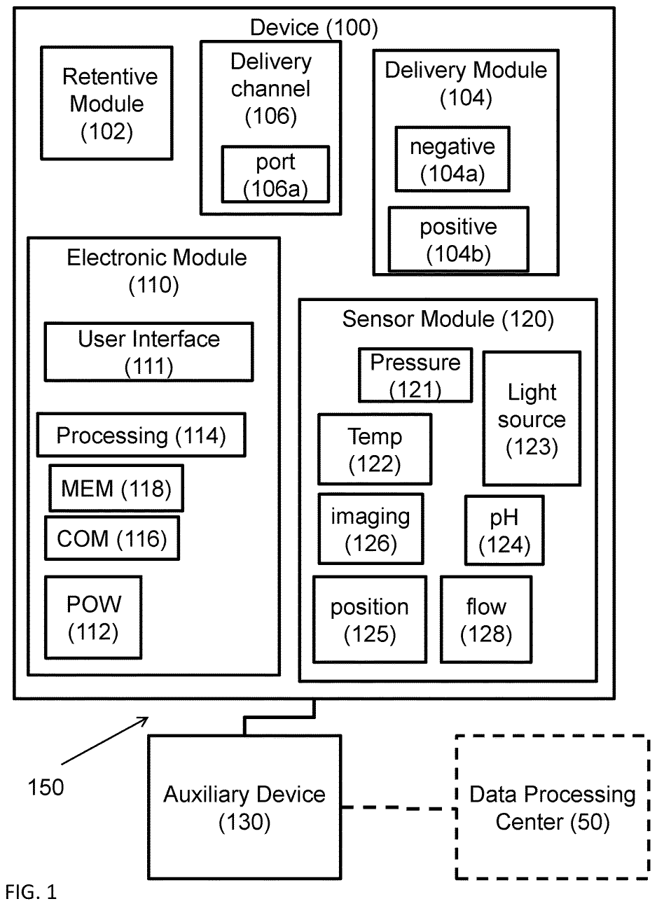

FIG. 1 shows a schematic block diagram of a controlled release intrauterine insemination device 100 and a system 150 thereof, according to embodiments of the present invention. Device 100 preferably utilized to facilitate delivery of an insemination fluid, for example a semen sample, to an intrauterine location so as to improve likelihood of fertilization. Device 100 may be configured to be used for human and/or veterinary use.

In some embodiments device 100 may be configured to be utilized for insemination of livestock for example including but not limited to horses, cows, sheep, goat, bovine, buffalo, or the like. In embodiments, device 100 may be configured to be utilized for insemination of wild and/or endangered animals.

In embodiments device 100 comprises a retentive module 102, a delivery module 104, delivery channel 106, electronics module 110 and a sensor module 120.

Device 100 comprises a retentive module 102 that is configured to be provided for holding and/or anchoring device 100 within the female anatomy, for example within the uterus and/or vagina. In embodiments retentive module 102 may be provided in the form of an expandable balloon and/or a stent like anchoring device. In embodiments retentive module 102 may be provided so as to retain and/or hold device 100 against the uterine wall and/or vaginal wall and/or cervix so as to allow fixation and/or positioning device 100 within the female anatomy.

In embodiments retentive module 102 may comprise at least one or more retentive members for example in the form of an inflatable balloon and/or a stent and/or a stent-like anchoring structure that may be placed for a period of time and thereafter removed.

In embodiments retentive module 102 may be comprise two individual retentive members for example a first distal retentive member may be placed within the uterus, past (distal) with respect to the cervix, and a second retentive member may be placed within the vaginal cavity, before (proximal) relative to the cervix.

In embodiments each retentive element may be formed from at least one or more cooperating members. For example, a single retentive member, such as an inflatable balloon, may be formed from at least two or more induvial inflatable balloons that cooperate with one another.

In some embodiments, a retentive member may be configured to be a housing for at least a portion of device 100. In some embodiments a proximal retentive member may be configured to house at least a portion of device 100.

In some embodiments at least a portion of retentive module 102 may be configured and/or shaped to accommodate and/or compliment additional tools and/or devices to facilitate an insemination procedure. For example, a portion of retentive module 102 may be configured to associate with gynecological tools to facilitate placement within the uterus, for example including but not limited to speculum or the like.

In embodiments device 100 comprises a delivery module 104 configured to deliver an insemination fluid, preferably comprising but not limited to a semen sample. In embodiments delivery module 104 may be configured to receive a semen sample by way of suctioning and/or up-taking a semen sample from a receptacle, in preparation for delivery within the female anatomy. In embodiments delivery module 104 may be configured to deliver a semen sample to a delivery site in the uterus and/or the intrauterine space by way of mobilizing and/or ejecting and/or discharging a semen sample from a receptacle of device to the delivery site.

In embodiments delivery module 104 may therefore comprise at least one or both of a negative pressure sub-module 104a, configured for generating suctioning and/or vacuum to facilitate sample uptake, and/or a positive pressure sub-module 104b configured for ejecting and/or delivering the semen sample to the intrauterine delivery site.

In embodiments delivery module 104 may be provided in the form of an electromechanical pump. In embodiments a controllable electromechanical pump may be provided so as to control the delivery rate such that device 100 may provide at least one of a slow release of the insemination fluid; a bolus release of the insemination fluid, or a combination thereof or the like.

In some embodiments delivery module 104 may be provided in optional forms for example including but is not limited to at least one or more of a motors, hydraulic motor, electromagnetic motor, piston, air piston, spring loaded movement, gear, linear spring, windings, electromagnet, pump, compressor, the like or any combination thereof.

In embodiments device 100 comprises a delivery channel 106 that is configured to delivery an insemination fluid and/or sample to the delivery site. In embodiments delivery channel 106 may be configured as an elongated tube that may directly uptake and/or receive a semen sample for delivery. In embodiments the delivery rate through channel 106 is most preferably controlled with by delivery module 104 so as to allow for at least one of slow release insemination and/or bolus insemination.

In embodiments delivery channel 106 may be configured in the form of a catheter or the like receptacle.

In embodiments at least a portion of channel 106 may feature at least one or more sensors of sensor module 120.

In embodiments preferably channel 106 is preferably fit with at least one or more retentive module 102 along its length.

In embodiments delivery channel 106 may be configured to have at least one or more exit ports 106a through which an insemination sample is delivered to the female uterine anatomy.

In some embodiments delivery channel 106 may be fit with a plurality of optional exit ports disposed along its length so as to preferably allow for controlling the location of the delivery of the insemination sample. For example, a first exit port 106a may be disposed at a distal end of the delivery channel 106, while a second exit port may be disposed along the length of channel 106 that is proximal to the distal end of channel 106, therein providing two optional delivery locations.

In embodiments channel 106 may be with a selector member for selecting a port 106a through which delivery is to be established.

In embodiments channel 106 may be fit with at least one or more sensor forming sensor module 120. In embodiments sensors forming sensor module 120 may be disposed about an exit port 106a of channel 106.

In embodiments device 100 comprises electronic module 110 comprising electronics circuitry, hardware, software to render device 100 functional and operational.

In embodiments electronics module 110 may comprise a plurality of optional sub-modules for example including but not limited to a power supply module 112, controller and/or processor module 114, user interface module 111, and memory module 118. In a preferred embodiments electronics module 110 may further comprise a communication module 116.

In embodiments electronics module 110 may be functionally associated with and/or comprise a sensor module 120.

In embodiments, User Interface (UI) module 111 may provide a user with means for interfacing with device 100 preferably via processor module 114. User interface 111 may be provided in the form of an activation button, audiovisual display, the like or any combination thereof. In embodiments, UI module 111 may be provided in optional forms for example including but not limited to buttons, dials, displays, alphanumeric display, touch screen, touch pad, buzzer, tactile pad, at least one light emitting diode (LED), at least one organic LED (OLED), speakers, microphone, or any combination thereof.

In embodiments processor module 114 provides the necessary processing hardware and/or software necessary to render device 100 functional. In embodiments controller and/or processor module 114 may provide for controlling any portion of device 100 and in particular delivery module 104 and sensor module 120. For example, processor 114 may be utilized to determine the status of a semen sample associated with channel 106 with at least one or more sensor of sensor module 120 so as to determine the delivery rate to be utilized with delivery module 104, for example a bolus delivery rate or a slow release delivery rate. Optionally processor module 114 and sensor module 120 may further facilitate determine which port 106a and/or where to deliver a sample along channel 106. For example, a non-limiting example, a sensed event with an optional sensor of sensor module 120 disposed adjacent to at least one port 106a, for example include but is not limited to sensing the pH in the immediate environment of port 106a may be utilized to determine where along channel 106 the best environment exist for the delivery of an insemination fluid and at which rate the delivery should be undertaken with delivery module 104, so as to optimize the likelihood of a successful procedure.

In embodiments power module 112 provides the necessary hardware and/or software to power device 100 therein rendering device 100 operational. Power module 112 may for example be provided in optional forms for example including but not limited to battery, rechargeable induction battery, induction coil, capacitors, super capacitors, inductors the like power source or any combination thereof.

In embodiments communication module 116 preferably provides the necessary hardware and/or software to facilitate communication for device 100 and in particular with auxiliary device 130 and/or data processing center 50 forming system 150. Optionally communication module 116 may be utilized to communicate with auxiliary devices 130, for example provided in the form of a processing and communication device such as a smartphone. In embodiments, an auxiliary device 130 may for example include but is not limited to a smartphone, mobile processing and communication device, imaging device, server, computer, healthcare service provider dedicated system, first respondent call center, health care call center, the like or any combination thereof.

In some embodiments communications module 116 may be utilize various communication protocols for example including but not limited to wireless communication, cellular communication, wired communication, near field communication, BLUETOOTH, ZIGBEE, optical communication, the like and/or any combination thereof.

In embodiments memory module 118 provides the necessary hardware and/or software to facilitate operations of device 100 by enabling storing and/or retrieving stored data and/or the like as is known in the art.

In embodiments sensor module 120 provides the necessary hardware and/or software to facilitate operations of at least one or more sensor(s) associated with device 100 to enable sensing various events in and around device 100 both internal and/or external to device 100. For example, in some embodiments sensor module 120 may be configured to sense the internal environment by sensing the environment in and around channel 106. In some embodiments, for example, sensor module 120 may be configured to sense the environment external to channel 106, for example, the intrauterine space and in particular the delivery site in the intrauterine space.

In embodiments sensor module 120 may comprise at least one or more sensor selected from the group consisting of: image sensor 126, temperature sensor 122, pH sensor 124, flow-meter 128, pulse oximeter, pressure sensor 121, acoustic sensor, microphone, positional sensor 125, light source 123 the like or any combination thereof.

In embodiments sensor module 120 may be disposed along any portion of device 100, and in along particular channel 106 and/or retentive module 102.

In embodiments image sensor 126 may be provided in optional forms for example including but not limited to camera that provides imagery of a distal end of channel 106. In some embodiments image sensor may be provided in the form of ultrasound sensor, that may optionally be disposed along a portion of retentive module 102.

In some embodiments at least a portion of channel 106 may feature an image sensor 126 in the form of a camera, for example for facilitating and/or determining placement and/or location of a sample within the intrauterine space by way of visual assistance with an image sensor 126 in the form of a camera.

In embodiments an image sensor 126, optionally in the form of a camera, may be configured to provide image processing and/or analysis so as to infer and/or obtain parametric data relating to the sample associated with device 100 via channel 106. For example, such image processing analysis may provide to determine at least one parameter for example including but not limited to: delivery site within the uterus, proper placement of device, sample volume, concentration of semen within sample, semen morphology, semen motility within sample, the like parameters associated with the sample or its contents, any combination thereof.

In embodiments the delivery channel 106 featuring at least one sensor and/or sensor module may comprise a pressure sensor 121, that is preferably disposed about a leading edge and/or distal end of the delivery channel 106 in order to facilitate proper placement of the device. Preferably pressure sensor 121 provides for ensuring that the placement is done accurately without perforating and/or damaging tissue at the delivery site. Preferably pressure sensor 121 allows for limiting the force and/or pressure applied during placement of the device.

In some embodiments the delivery channel 106 may feature a light source 123 for facilitating placement of the device at the treatment site. Optionally the light source 123 may be provided in optional forms for example including but not limited to a light emitting diode ‘LED’ disposed at a portion of the distal end of the delivery channel 106. Optionally the light source may be provided in the form of an optic fiber and associated light source such that the optic fiber tip is disposed at the distal end of the delivery channel while the associated light source is disposed at a proximal portion of the delivery channel 106.

In some embodiments sensor module 120 may comprise at least one position sensor 125 configured to identify the position a delivery channel 106 so as to convey the spatial location of a semen sample within the intrauterine space. In embodiments position sensor 125 may be provided in optional forms for example including but not limited to optical sensors, mechanical sensors, electromagnetic sensor, induction sensor, magnetic based sensors or the like sensor provided for determining and the relative position of sample and/or delivery channel 106 within the uterus.

In embodiments system 150 comprises device 100 that is in communication with an auxiliary device 130 and/or data processing center 50. In embodiments data processing center 50 may be provided in optional forms for example including but not limited to a server, computer, smartphone, or the like processing and communication device capable of receiving and/or exchanging data from device 100 so as to process the data. In embodiments, processing center 50 is preferably utilized to apply big data algorithms and/or the like artificial intelligence algorithms to analyze data received from device 100. Optionally communication between processing center 50 and device 100 is facilitated with communication module 116 utilizing any communication protocol for example including wireless, cellular, wired, near field, the like or any combination thereof.

In embodiments system 150 and/or device 100 and/or auxiliary device 130 and/or data center 50 may communicate with additional auxiliary devices to undertake processing of data associated with system 150 and/or device 100. For example, such auxiliary device communication devices may for example include but is not limited to a smartphone, mobile processing and communication device, imaging device, server, computer, healthcare service provider dedicated system, first respondent call center, health care call center, the like or any combination thereof.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It should be noted that where reference numerals appear in the claims, such numerals are included solely or the purpose of improving the intelligibility of the claims and are no way limiting on the scope of the claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

While the invention has been described with respect to a limited number of embodiments, it will be appreciated that many variations, modifications and other applications of the invention may be made.

1) A controlled release intrauterine insemination device the device comprising, a retentive module, a delivery channel a delivery module, an electronics module and a sensor module wherein said delivery module is controlled based on data captured with said sensor module to control a delivery rate of a sample associated with said channel so as to determine sample delivery parameters.

2) The device of claim 1 wherein said retentive module is an inflatable balloon or a stent-like expandable anchor.

3) The device of claim 1 wherein said retentive module comprises at least two or more retentive members selected from an inflatable balloon or a stent-like anchor.

4) (canceled)

5) The device of claim 3 wherein said retentive members are disposed along different locations of said delivery channel.

6) The device of claim 5 wherein a first retentive member is disposed adjacent to a distal end of said delivery channel and a second retentive member is disposed adjacent to a proximal end of said delivery channel.

7) The device of claim 6 wherein said second retentive member is configured in the form of a device housing.

8) (canceled)

9) (canceled)

10) The device of claim 1 wherein said delivery channel has a distal end that is distal to a distal end of said retentive module.

11) The device of claim 1 wherein said delivery channel is associated with at least one or more sensors of said sensor module wherein said at least one or more sensors is disposed along said delivery channel.

12) The device of claim 11 wherein said at least one or more sensors are disposed at a location selected from:
a) along an end of said delivery channel; or
b) adjacent to a distal end of said delivery channel; or
c) along a portion of a distal end of said delivery channel; or
d) along the length of said delivery channel; or
e) any combination thereof.

13) (canceled)

14) (canceled)

15) (canceled)

16) The device of claim 11 wherein two of the same sensor types are disposed along two different locations along said delivery channel.

17) The device of claim 11 wherein said sensor is a pressure sensor or a light source.

18) (canceled)

19) The device of claim 1 wherein said delivery channel comprises at least two or more delivery ports.

20) The device of claim 19 wherein at least one delivery port is disposed along a distal end of said delivery channel.

21) The device of claim 19 wherein a second delivery port is disposed along the length of said delivery channel.

22) The device of claim 19 wherein at least one or more sensor is disposed adjacent to each of said delivery ports.

23) The device of claim 1 wherein said sensor module comprises at least one sensor selected from: image sensor, pH sensor, flow sensor, temperature sensor, pressure sensor, light source, positional sensor, microphone, acoustic sensor, pulse oximeter, flow meter, or any combination thereof.

24) The device of claim 1 wherein said delivery module is provided in the form of an electromechanical pump.

25) The device of claim 1 wherein said delivery module is configured to generate either positive pressure or negative pressure adjacent to said delivery port.

26) A controlled release intrauterine insemination system comprising the device of claim 1 that is in wireless communication with at least one or more processing device selected from: an auxiliary-processing and communication device and/or a data processing center.

27) (canceled)

28) A method for controlling the delivery rate of an insemination sample with the device of claim 1, so as to determine the delivery parameters of an insemination-sample, the parameters selected from at least one of:
a) determining delivery site withing the intrauterine space; or
b) determining rate of sample delivery; or
c) determining the timing of sample delivery; or
d) determining the sample delivery ejection speed; or
e) determining the sample delivery ejection force.
